(19) United States
(12) Patent Application Publication
(10) Pub. No.: US 2021/0069127 A1
(43) Pub. Date: Mar. 11, 2021

(54) Multi-Dose Concentrated Liquid Diphenhydramine HCl Compositions and Packaged Multi-Dose Liquid Diphenhydramine HCl Formulations

(71) Applicant: DXM Pharmaceuticals, Inc., Katy, TX (US)

(72) Inventors: Mark Lawrence Thuesen, Conroe, TX (US); Steve Shane Sanders, Lake Charles, LA (US); Steve J. Bannister, Tampa, FL (US)

(73) Assignee: DXM Pharmaceuticals, Inc., Katy, TX (US)

(21) Appl. No.: 16/896,259

(22) Filed: Jun. 9, 2020

Related U.S. Application Data
(63) Continuation-in-part of application No. 15/936,129, filed on Mar. 26, 2018.
Application No. 15/936,129 is a continuation-in-part of application No. 62/606,153, filed on Jan. 26, 2018.
(60) Provisional application No. 62/931,830, filed on Nov. 7, 2019.
(60) Provisional application No. 62/859,129, filed on Jun. 9, 2019.
(60) Provisional application No. 62/476,921, filed on Mar. 27, 2017.

(51) Int. Cl. A61K 31/138; A61K 9/00

(52) U.S. Cl. CPC A61K 31/138; A61K 47/10; A61K 9/0095

(57) ABSTRACT

Liquid formulations or syrup comprise an over-the-counter API ingredient or a dietary supplement ingredient. The ingredients are present in concentrated amounts. Multiple doses of the syrups may be packaged in relatively small bottles, such as bottles with a capacity of 100 ml or less.

### FIELD OF THE INVENTION

The present invention relates to packaged over-the-counter concentrated liquid drug compositions and concentrated liquid dietary supplement compositions, and more particularly, to such formulations and multi-dose delivery systems for such formulations.

### BACKGROUND OF THE INVENTION

Over-the-counter (“OTC”) drugs are sold directly to consumers and do not require a prescription from a healthcare professional. They are generally recognized as safe and effective for their intended uses and allow consumers to self-diagnose and treat a variety of conditions. OTC drugs are available in many different forms. Common forms include topicals, such as creams and ointment which are intended to be applied to the skin, and orals, such as pills and liquids that are taken by mouth. They may be used to treat many different conditions. Most commonly, they are sold to relieve aches and pains, to relieve fever, congestion, coughing and other cold and flu symptoms, to relieve allergic reactions, and to relieve gas, acid reflux, and other gastrointestinal symptoms.

OTC drugs are a large market. Global sales of over-the-counter (“OTC”) drugs likely exceed $60 billion per year. The market in the United States represents over 30% of the worldwide market with approximately $20 billion in annual sales. The consensus is that such sales will continue to expand significantly, especially in developing markets.

Despite an expanding market, however, competition in the OTC market is fierce. Hundreds, if not thousands of new OTC drugs are introduced every year. Unlike new drugs, OTC drugs typically do not require any individual review by regulatory agencies and may be introduced more quickly and with less expense. In the United States, for example, the United States Food and Drug Administration (“FDA”) has established “monographs” covering over 200 different active ingredients classified into 26 therapeutic categories.

The monographs are essentially rule books. They specify acceptable active pharmaceutical ingredients (“API”), inactive ingredients, uses or “indications,” doses, formulations, testing, and labeling requirements that are intended to ensure that an OTC drug is labeled properly. If an API meets the standards in a monograph and complies with other applicable FDA regulations, the drug will be considered safe and effective and may be marketed without a lengthy and costly individual review.

The 30 most common OTC APIs that are offered in the USA are the following: acetaminophen, aluminium hydroxide, aspirin, bisacodyl, Bismuth subsalicylate, caffeine, calcium carbonate, centirizine HCl, choline salicylate, cimetidine, dextromethorphan HBr, dextromethorphan polistirex, dimenhydrinate, diphenhydramine HCl, doxylamine succinate, esomeprazole magnesium, famotidine, fexofenadine HCl, guaifenesin, ibuprofen, levocetirizine dihydrochloride, loperamide HCl, loratadine, magnesium hydroxide, naproxen sodium, nicotine polacrilex, omeprazole, phenylephrine HCl, ranitidine HCl, and simethicone (“the Top 30 OTC APIs”).

They are available, either alone or in combination with other APIs, in more than 3,000 OTC formulations, primarily in the form of pills or liquids to be taken orally. Whether alone or formulated with other APIs, OTC formulations are almost exclusively distributed in multi-dose packages. Approximately 1 billion bottles of oral liquid OTC formulations are distributed annually in the USA, equaling approximately 10 billion doses. Approximately 94 billion doses are in the form of pills, thus liquid doses represent about 9.6% of all doses in the USA. Because liquid doses are at least two times more expensive than pill doses, however, liquid doses represent about 18% of overall sales in the USA.

OTC APIs tend to have a bitter taste when packaged into liquid formulations. That bitterness can make consumers hesitant to follow recommended dosages, or refusal to consume liquid formulations. It also can cause consumers to prefer less bitter formulations diluted by higher amounts of water. The primary method of avoiding the bitter taste is to formulate the oral liquid solution with relatively low concentrations of APIs. However, diluting a bitter tasting API with higher amounts of water simply increases the amount of bitter tasting liquid ingested. Thus, OTC formulations with APIs typically include flavoring agents or agents designed to mask the bitterness. Various other approaches, such as increasing the viscosity and adjusting the pH of the formulation also are known to reduce the level of perceived bitterness in certain formulations.

There are many challenges in marketing and distributing an OTC drug. Manufacturers are increasingly called upon to package and promote their products to meet ever more particularized consumer and retail demands. Such customization may take many different forms. A manufacturer may be required, for example, to provide an OTC drug in both pill and liquid form, in different strengths, in various combinations with other OTC drugs, in multiple package sizes and formats, in adult and child formulations, and even in different flavors and “sugar-free.” Pfizer, for example, currently markets 20 different Robitussin® liquid cough and cold formulations all in various sizes.

Even under the best of circumstances it is difficult for an OTC product to find room on a retailer's shelf. Retailers may be more than willing to provide their customers with a variety of choices, but shelf space is limited. Products often are displayed within inches of each other on store shelves, and there is little or no cost for a consumer to switch brands. A relatively few brands, such as Robitussin and Mucinex, have built a loyal base of consumers, but most brands fight daily for their survival. Even famous, established brands face challenges from generics and store brands and must be vigilant in protecting their market share.

Consequently, over 95% of OTC drug brands are distributed through relatively large retailers, such as drugstores and grocery stores. A typical drugstore may have about 11,000 square feet (sf) of retail space. An average grocery store may have about 45,000 sf, with superstores having up to about 200,000 sf. While such stores sell a large number of SKUs (Stock Keeping Units) in many different product categories, they still devote relatively large amounts of shelving to OTC drug formulations. A typical drug store, for example, may have 100 to 150 feet of shelf space devoted to cough, cold, and flu formulations alone. Even with relatively large sections, however, even a famous brand like Robitussin may only be able to display half of its formulations in a couple of sizes. Most OTC drug brands, even famous brands, have not been able to successfully generate meaningful revenue from their products through convenience stores or the Internet.

Although there now are smaller “kiosk” and larger “hyper” convenience stores, traditional convenience stores have been in the range of 2,400 to 2,500 square feet. They not only stock many fewer SKUs, but much of their space is devoted to the products where they derive their greatest sales. Excluding gas, the top product categories are lottery tickets, cigarettes, non-alcoholic packaged beverages, food service, beer, tobacco products other than cigarettes, candy, salty snacks, general merchandise, fluid milk products, and packaged sweet snacks. Those top categories account for the vast majority of sales in convenience stores, with the top five categories accounting for approximately 80% of sales.

There is very little space left for OTC drugs and other products. A “well stocked” convenience store will average less than 5 feet of shelf space stocking 20 to 30 SKUs of OTC drugs. Most of those SKUs are typically pills as many doses of OTC pills may be provided in relatively small packaging. Multi-dose liquid OTC drugs typically require much larger packaging. Fewer multi-dose liquid OTC drug SKUs may be displayed in the same shelf space. Consequently, some OTC manufacturers have provided single dose packaging for liquid OTC drugs. Such packaging necessarily can be much smaller. Nevertheless, the bulky packaging required for multi-dose liquid OTC drugs has made it difficult for manufacturers to distribute through convenience stores. At most, a convenience store may limit its inventory to 2 to 5 multi-dose liquid OTC formulations. Some convenience stores stock pill and tablet OTC drugs exclusively.

Most illnesses are unexpected, and consumers tend to purchase OTC drugs when they become ill. That creates additional barriers to distributing OTC drugs over the Internet or in convenience stores. Consumers are unwilling to wait for their OTC drugs to be delivered. Even one-day delivery is unacceptably long. They want immediate relief. Immediate relief also likely cannot be found at a typical convenience store. Many convenience stores may stock cough drops and antacids. Smoking-related coughing and stomach indigestion are quite common and are more predictable. Because they are small and have limited shelf space, however, convenience stores rarely carry a large selection of OTC drugs. They will not be an option for consumers suffering from other symptoms.

Shelf life can present additional challenges in distributing OTC formulations. Most retailers require a minimum shelf life of at least 90 days. Many retailers, especially smaller retailers such as convenience stores that may have less turnover, may prefer even longer shelf lives, for example, a year or more. It may be difficult to provide assurances that OTC formulations will have acceptable shelf lives.

The active ingredients in OTC formulations are stable to varying degrees. They may degrade over time. Likewise, the formulation components are compatible to varying degrees and may separate, agglomerate, or otherwise degrade. Degradation and in turn shelf like can be affected greatly by the temperature at which the formulation is stored. Light, air, and humidity passing through the packaging also can degrade the formulation.

Similar challenges are presented in manufacturing and distributing dietary supplement formulations. Many dietary ingredients are bitter tasting and limit consumer acceptance and use in liquid formulations. It also may be difficult to acquire and maintain shelf space in retail outlets, especially convenience stores. Shelf life also can be a problem.

The statements in this section are intended to provide background information related to the invention disclosed and claimed herein. Such information may or may not constitute prior art. It will be appreciated from the foregoing, however, that there remains a need for new and improved packaged, multi-dose concentrated liquid OTC drug and dietary supplement compositions, especially those with extended shelf lives. Such disadvantages and others inherent in the prior art are addressed by various aspects and embodiments of the subject invention.

### SUMMARY OF THE INVENTION

The subject invention, in its various aspects and embodiments, relates generally to packaged over-the-counter concentrated liquid compositions containing an active, OTC ingredient or a dietary supplement ingredient. The invention encompasses various embodiments and aspects, some of which are specifically described and illustrated herein.

One aspect of the invention provides for multi-dose packaged over-the-counter liquid Top 30 APIs. The packaged syrups comprise a bottle containing about 75 ml or less of a liquid formulation of the Top 30 APIs, but could have as much as 354 ml or more. The liquid formulation provides at least two doses of one, or combinations, of the Top 30 APIs.

Other embodiments provide such packaged syrups where the liquid formulation provides from about 4 to about 50 doses.

The concentration of APIs into liquid formulations is limited by the solubility of the API into the liquid excipient and the FDA approved dosage amount in milligrams needed to provide the FDA approved desired relief. The concentration of APIs is further limited by the liquid excipient temperature and other inactive ingredients added to the formulation. Making the concentration of APIs even more difficult to predict is the requirement for stability testing and freeze thaw cycling.

Yet other such embodiments provide doses comprising an amount of APIs specified by Regulatory Guidelines or by the United States FDA Final Monograph for the API.

In other aspects and embodiments, the invention provides a dextromethorphan HBr API composition. The composition comprises water, dextromethorphan HBr in amounts greater than about 7 mg/ml, and sugar in amounts greater than about 200 mg/ml, preferably from about 300 to about 700 mg/ml. Other embodiments of the composition comprise water, dextromethorphan HBr in amounts greater than about 7 mg/ml, and sugar substitutes in amounts greater than about 2 mg/ml, preferably from about 5 to about 20 mg/ml. Additional embodiments of the composition comprise water, dextromethorphan HBr in amounts greater than about 7 mg/ml, and sugar in amounts greater than about 200 mg/ml and sugar substitutes in amounts greater than about 2 mg/ml.

In other aspects and embodiments, the invention provides a dextromethorphan polistirex API composition. The composition comprises water, dextromethorphan polistirex in amounts greater than about 9 mg/ml, and sugar in amounts greater than about 200 mg/ml, preferably from about 300 to about 700 mg/ml. Other embodiments of the composition comprise water, dextromethorphan polistirex in amounts greater than about 9 mg/ml, and sugar substitutes in amounts greater than about 2 mg/ml, preferably from about 5 to about 20 mg/ml. Additional embodiments of the composition comprise water, dextromethorphan polistirex in amounts greater than about 9 mg/ml, and sugar in amounts greater than about 200 mg/ml and sugar substitutes in amounts greater than about 2 mg/ml.

In other aspects and embodiments, the invention provides an acetaminophen API composition. The composition comprises water, acetaminophen in amounts greater than about 50 mg/ml, and sugar in amounts greater than about 200 mg/ml, preferably from about 300 to about 700 mg/ml. Other embodiments of the composition comprise water, acetaminophen in amounts greater than about 50 mg/ml, and sugar substitutes in amounts greater than about 2 mg/ml, preferably from about 5 to about 20 mg/ml. Additional embodiments of the composition comprise water, acetaminophen in amounts greater than about 50 mg/ml, and sugar in amounts greater than about 200 mg/ml and sugar substitutes in amounts greater than about 2 mg/ml.

In other aspects and embodiments, the invention provides an aluminum hydroxide API composition. The composition comprises water, aluminum hydroxide in amounts greater than about 120 mg/ml, and sugar in amounts greater than about 200 mg/ml, preferably from about 300 to about 700 mg/ml. Other embodiments of the composition comprise water, aluminum hydroxide in amounts greater than about 120 mg/ml, and sugar substitutes in amounts greater than about 2 mg/ml, preferably from about 5 to about 20 mg/ml. Additional embodiments of the composition comprise water, aluminum hydroxide in amounts greater than about 120 mg/ml, and sugar in amounts greater than about 200 mg/ml and sugar substitutes in amounts greater than about 2 mg/ml.

In other aspects and embodiments, the invention provides a bismuth subsalicylate API composition. The composition comprises water, bismuth subsalicylate in amounts greater than about 26 mg/ml, and sugar in amounts greater than about 200 mg/ml, preferably from about 300 to about 700 mg/ml. Other embodiments of the composition comprise water, bismuth subsalicylate in amounts greater than about 26 mg/ml, and sugar substitutes in amounts greater than about 2 mg/ml, preferably from about 5 to about 20 mg/ml. Additional embodiments of the composition comprise water, bismuth subsalicylate in amounts greater than about 26 mg/ml, and sugar in amounts greater than about 200 mg/ml and sugar substitutes in amounts greater than about 2 mg/ml.

In other aspects and embodiments, the invention provides a caffeine API composition. The composition comprises water, caffeine in amounts greater than about 6 mg/ml, and sugar in amounts greater than about 200 mg/ml, preferably from about 300 to about 700 mg/ml. Other embodiments of the composition comprise water, caffeine in amounts greater than about 6 mg/ml, and sugar substitutes in amounts greater than about 2 mg/ml, preferably from about 5 to about 20 mg/ml. Additional embodiments of the composition comprise water, caffeine in amounts greater than about 6 mg/ml, and sugar in amounts greater than about 200 mg/ml and sugar substitutes in amounts greater than about 2 mg/ml.

In other aspects and embodiments, the invention provides a centirizine HCl API composition. The composition comprises water, centirizine HCl in amounts greater than about 1.5 mg/ml, and sugar in amounts greater than about 200 mg/ml, preferably from about 300 to about 700 mg/ml. Other embodiments of the composition comprise water, centirizine HCl in amounts greater than about 1.5 mg/ml, and sugar substitutes in amounts greater than about 2 mg/ml, preferably from about 5 to about 20 mg/ml. Additional embodiments of the composition comprise water, centirizine HCl in amounts greater than about 1.5 mg/ml, and sugar in amounts greater than about 200 mg/ml and sugar substitutes in amounts greater than about 2 mg/ml.

In other aspects and embodiments, the invention provides a choline salicylate API composition. The composition comprises water, choline salicylate in amounts greater than about 23 mg/ml, and sugar in amounts greater than about 200 mg/ml, preferably from about 300 to about 700 mg/ml. Other embodiments of the composition comprise water, choline salicylate in amounts greater than about 23 mg/ml, and sugar substitutes in amounts greater than about 2 mg/ml, preferably from about 5 to about 20 mg/ml. Additional embodiments of the composition comprise water, choline salicylate in amounts greater than about 23 mg/ml, and sugar in amounts greater than about 200 mg/ml and sugar substitutes in amounts greater than about 2 mg/ml.

In other aspects and embodiments, the invention provides a codeine (also includes codeine phosphate) API composition. The composition comprises water, codeine in amounts greater than about 7 mg/ml, and sugar in amounts greater than about 200 mg/ml, preferably from about 300 to about 700 mg/ml. Other embodiments of the composition comprise water, codeine in amounts greater than about 7 mg/ml, and sugar substitutes in amounts greater than about 2 mg/ml, preferably from about 5 to about 20 mg/ml. Additional embodiments of the composition comprise water, codeine in amounts greater than about 7 mg/ml, and sugar in amounts greater than about 200 mg/ml and sugar substitutes in amounts greater than about 2 mg/ml.

In other aspects and embodiments, the invention provides a diphenhydramine HC API composition. The composition comprises water, diphenhydramine HCl in amounts greater than about 4 mg/ml, and sugar in amounts greater than about 200 mg/ml, preferably from about 300 to about 700 mg/ml. Other embodiments of the composition comprise water, diphenhydramine HCl in amounts greater than about 4 mg/ml, and sugar substitutes in amounts greater than about 2 mg/ml, preferably from about 5 to about 20 mg/ml. Additional embodiments of the composition comprise water, diphenhydramine HCl in amounts greater than about 4 mg/ml, and sugar in amounts greater than about 200 mg/ml and sugar substitutes in amounts greater than about 2 mg/ml.

In other aspects and embodiments, the invention provides a doxylamine succinate API composition. The composition comprises water, doxylamine succinate in amounts greater than about 1.5 mg/ml, and sugar in amounts greater than about 200 mg/ml, preferably from about 300 to about 700 mg/ml. Other embodiments of the composition comprise water, doxylamine succinate in amounts greater than about 1.5 mg/ml, and sugar substitutes in amounts greater than about 2 mg/ml, preferably from about 5 to about 20 mg/ml. Additional embodiments of the composition comprise water, doxylamine succinate in amounts greater than about 1.5 mg/ml, and sugar in amounts greater than about 200 mg/ml and sugar substitutes in amounts greater than about 2 mg/ml.

In other aspects and embodiments, the invention provides a fexofenadine HCl API composition. The composition comprises water, fexofenadine HCl in amounts greater than about 9 mg/ml, and sugar in amounts greater than about 200 mg/ml, preferably from about 300 to about 700 mg/ml. Other embodiments of the composition comprise water, fexofenadine HCl in amounts greater than about 9 mg/ml, and sugar substitutes in amounts greater than about 2 mg/ml, preferably from about 5 to about 20 mg/ml. Additional embodiments of the composition comprise water, fexofenadine HCl in amounts greater than about 9 mg/ml, and sugar in amounts greater than about 200 mg/ml and sugar substitutes in amounts greater than about 2 mg/ml.

In other aspects and embodiments, the invention provides a guaifenesin API composition. The composition comprises water, guaifenesin in amounts greater than about 45 mg/ml, and sugar in amounts greater than about 200 mg/ml, preferably from about 300 to about 700 mg/ml. Other embodiments of the composition comprise water, guaifenesin in amounts greater than about 45 mg/ml, and sugar substitutes in amounts greater than about 2 mg/ml, preferably from about 5 to about 20 mg/ml. Additional embodiments of the composition comprise water, guaifenesin in amounts greater than about 45 mg/ml, and sugar in amounts greater than about 200 mg/ml and sugar substitutes in amounts greater than about 2 mg/ml.

In other aspects and embodiments, the invention provides an ibuprofen API composition. The composition comprises water, ibuprofen in amounts greater than about 60 mg/ml, and sugar in amounts greater than about 200 mg/ml, preferably from about 300 to about 700 mg/ml. Other embodiments of the composition comprise water, ibuprofen in amounts greater than about 60 mg/ml, and sugar substitutes in amounts greater than about 2 mg/ml, preferably from about 5 to about 20 mg/ml. Additional embodiments of the composition comprise water, ibuprofen in amounts greater than about 60 mg/ml, and sugar in amounts greater than about 200 mg/ml and sugar substitutes in amounts greater than about 2 mg/ml.

In other aspects and embodiments, the invention provides a loratadine API composition. The composition comprises water, loratadine in amounts greater than about 1.5 mg/ml, and sugar in amounts greater than about 200 mg/ml, preferably from about 300 to about 700 mg/ml. Other embodiments of the composition comprise water, loratadine in amounts greater than about 1.5 mg/ml, and sugar substitutes in amounts greater than about 2 mg/ml, preferably from about 5 to about 20 mg/ml. Additional embodiments of the composition comprise water, loratadine in amounts greater than about 1.5 mg/ml, and sugar in amounts greater than about 200 mg/ml and sugar substitutes in amounts greater than about 2 mg/ml.

In other aspects and embodiments, the invention provides a magnesium hydroxide API composition. The composition comprises water, magnesium hydroxide in amounts greater than about 120 mg/ml, and sugar in amounts greater than about 200 mg/ml, preferably from about 300 to about 700 mg/ml. Other embodiments of the composition comprise water, magnesium hydroxide in amounts greater than about 120 mg/ml, and sugar substitutes in amounts greater than about 2 mg/ml, preferably from about 5 to about 20 mg/ml. Additional embodiments of the composition comprise water, magnesium hydroxide in amounts greater than about 120 mg/ml, and sugar in amounts greater than about 200 mg/ml and sugar substitutes in amounts greater than about 2 mg/ml.

In other aspects and embodiments, the invention provides a phenylephrine HCl API composition. The composition comprises water, phenylephrine HCl in amounts greater than about 1 mg/ml, and sugar in amounts greater than about 200 mg/ml, preferably from about 300 to about 700 mg/ml. Other embodiments of the composition comprise water, phenylephrine HCl in amounts greater than about 1 mg/ml, and sugar substitutes in amounts greater than about 2 mg/ml, preferably from about 5 to about 20 mg/ml.

Additional embodiments of the composition comprise water, phenylephrine HCl in amounts greater than about 1 mg/ml, and sugar in amounts greater than about 200 mg/ml and sugar substitutes in amounts greater than about 2 mg/ml.

In other aspects and embodiments, the invention provides a simethicone API composition. The composition comprises water, simethicone in amounts greater than about 100 mg/ml, and sugar in amounts greater than about 200 mg/ml, preferably from about 300 to about 700 mg/ml. Other embodiments of the composition comprise water, simethicone in amounts greater than about 100 mg/ml, and sugar substitutes in amounts greater than about 2 mg/ml, preferably from about 5 to about 20 mg/ml. Additional embodiments of the composition comprise water, simethicone in amounts greater than about 100 mg/ml, and sugar in amounts greater than about 200 mg/ml and sugar substitutes in amounts greater than about 2 mg/ml.

In other aspects and embodiments, the invention provides an aspirin API composition. The composition comprises water, aspirin in amounts greater than about 750 mg/ml, and sugar in amounts greater than about 200 mg/ml, preferably from about 300 to about 700 mg/ml. Other embodiments of the composition comprise water, aspirin in amounts greater than about 750 mg/ml, and sugar substitutes in amounts greater than about 2 mg/ml, preferably from about 5 to about 20 mg/ml. Additional embodiments of the composition comprise water, aspirin in amounts greater than about 750 mg/ml, and sugar in amounts greater than about 200 mg/ml and sugar substitutes in amounts greater than about 2 mg/ml.

In other aspects and embodiments, the invention provides a bisacodyl API composition. The composition comprises water, bisacodyl in amounts greater than about 15 mg/ml, and sugar in amounts greater than about 200 mg/ml, preferably from about 300 to about 700 mg/ml. Other embodiments of the composition comprise water, bisacodyl in amounts greater than about 15 mg/ml, and sugar substitutes in amounts greater than about 2 mg/ml, preferably from about 5 to about 20 mg/ml. Additional embodiments of the composition comprise water, bisacodyl in amounts greater than about 15 mg/ml, and sugar in amounts greater than about 200 mg/ml and sugar substitutes in amounts greater than about 2 mg/ml.

In other aspects and embodiments, the invention provides a calcium carbonate API composition. The composition comprises water, calcium carbonate in amounts greater than about 3,000 mg/ml, and sugar in amounts greater than about 200 mg/ml, preferably from about 300 to about 700 mg/ml. Other embodiments of the composition comprise water, calcium carbonate in amounts greater than about 3,000 mg/ml, and sugar substitutes in amounts greater than about 2 mg/ml, preferably from about 5 to about 20 mg/ml. Additional embodiments of the composition comprise water, calcium carbonate in amounts greater than about 3,000 mg/ml, and sugar in amounts greater than about 200 mg/ml and sugar substitutes in amounts greater than about 2 mg/ml.

In other aspects and embodiments, the invention provides a cimetidine API composition. The composition comprises water, cimetidine in amounts greater than about 300 mg/ml, and sugar in amounts greater than about 200 mg/ml, preferably from about 300 to about 700 mg/ml. Other embodiments of the composition comprise water, cimetidine in amounts greater than about 300 mg/ml, and sugar substitutes in amounts greater than about 2 mg/ml, preferably from about 5 to about 20 mg/ml. Additional embodiments of the composition comprise water, cimetidine in amounts greater than about 300 mg/ml, and sugar in amounts greater than about 200 mg/ml and sugar substitutes in amounts greater than about 2 mg/ml.

In other aspects and embodiments, the invention provides a dimenhydrinate API composition. The composition comprises water, dimenhydrinate in amounts greater than about 38 mg/ml, and sugar in amounts greater than about 200 mg/ml, preferably from about 300 to about 700 mg/ml. Other embodiments of the composition comprise water, dimenhydrinate in amounts greater than about 38 mg/ml, and sugar substitutes in amounts greater than about 2 mg/ml, preferably from about 5 to about 20 mg/ml. Additional embodiments of the composition comprise water, dimenhydrinate in amounts greater than about 38 mg/ml, and sugar in amounts greater than about 200 mg/ml and sugar substitutes in amounts greater than about 2 mg/ml.

In other aspects and embodiments, the invention provides an esomeprazole magnesium API composition. The composition comprises water, esomeprazole magnesium in amounts greater than about 30 mg/ml, and sugar in amounts greater than about 200 mg/ml, preferably from about 300 to about 700 mg/ml. Other embodiments of the composition comprise water, esomeprazole magnesium in amounts greater than about 30 mg/ml, and sugar substitutes in amounts greater than about 2 mg/ml, preferably from about 5 to about 20 mg/ml. Additional embodiments of the composition comprise water, esomeprazole magnesium in amounts greater than about 30 mg/ml, and sugar in amounts greater than about 200 mg/ml and sugar substitutes in amounts greater than about 2 mg/ml.

In other aspects and embodiments, the invention provides a famotidine API composition. The composition comprises water, famotidine in amounts greater than about 30 mg/ml, and sugar in amounts greater than about 200 mg/ml, preferably from about 300 to about 700 mg/m. Other embodiments of the composition comprise water, famotidine in amounts greater than about 30 mg/ml, and sugar substitutes in amounts greater than about 2 mg/ml, preferably from about 5 to about 20 mg/ml. Additional embodiments of the composition comprise water, famotidine in amounts greater than about 30 mg/ml, and sugar in amounts greater than about 200 mg/ml and sugar substitutes in amounts greater than about 2 mg/ml.

In other aspects and embodiments, the invention provides a levocetirizine dihydrochloride API composition. The composition comprises water, levocetirizine dihydrochloride in amounts greater than about 7.5 mg/ml, and sugar in amounts greater than about 200 mg/ml, preferably from about 300 to about 700 mg/ml. Other embodiments of the composition comprise water, levocetirizine dihydrochloride in amounts greater than about 7.5 mg/ml, and sugar substitutes in amounts greater than about 2 mg/ml, preferably from about 5 to about 20 mg/ml. Additional embodiments of the composition comprise water, levocetirizine dihydrochloride in amounts greater than about 7.5 mg/ml, and sugar in amounts greater than about 200 mg/ml and sugar substitutes in amounts greater than about 2 mg/ml.

In other aspects and embodiments, the invention provides a loperamide HCl API composition. The composition comprises water, loperamide HCl in amounts greater than about 6 mg/ml, and sugar in amounts greater than about 200 mg/ml, preferably from about 300 to about 700 mg/ml. Other embodiments of the composition comprise water, loperamide HCl in amounts greater than about 6 mg/ml, and sugar substitutes in amounts greater than about 2 mg/ml, preferably from about 5 to about 20 mg/m. Additional embodiments of the composition comprise water, loperamide HCl in amounts greater than about 6 mg/ml, and sugar in amounts greater than about 200 mg/ml and sugar substitutes in amounts greater than about 2 mg/ml.

In other aspects and embodiments, the invention provides a naproxen sodium API composition. The composition comprises water, naproxen sodium in amounts greater than about 330 mg/ml, and sugar in amounts greater than about 200 mg/ml, preferably from about 300 to about 700 mg/ml. Other embodiments of the composition comprise water, naproxen sodium in amounts greater than about 330 mg/ml, and sugar substitutes in amounts greater than about 2 mg/ml, preferably from about 5 to about 20 mg/ml. Additional embodiments of the composition comprise water, naproxen sodium in amounts greater than about 330 mg/ml, and sugar in amounts greater than about 200 mg/ml and sugar substitutes in amounts greater than about 2 mg/ml.

In other aspects and embodiments, the invention provides an omeprazole API composition. The composition comprises water, omeprazole in amounts greater than about 30 mg/ml, and sugar in amounts greater than about 200 mg/ml, preferably from about 300 to about 700 mg/ml. Other embodiments of the composition comprise water, omeprazole in amounts greater than about 30 mg/ml, and sugar substitutes in amounts greater than about 2 mg/ml, preferably from about 5 to about 20 mg/ml. Additional embodiments of the composition comprise water, omeprazole in amounts greater than about 30 mg/ml, and sugar in amounts greater than about 200 mg/ml and sugar substitutes in amounts greater than about 2 mg/ml.

In other aspects and embodiments, the invention provides a ranitidine HCl API composition. The composition comprises water, ranitidine HCl in amounts greater than about 250 mg/ml, and sugar in amounts greater than about 200 mg/ml, preferably from about 300 to about 700 mg/ml. Other embodiments of the composition comprise water, ranitidine HCl in amounts greater than about 250 mg/ml, and sugar substitutes in amounts greater than about 2 mg/ml, preferably from about 5 to about 20 mg/ml. Additional embodiments of the composition comprise water, ranitidine HCl in amounts greater than about 250 mg/ml, and sugar in amounts greater than about 200 mg/ml and sugar substitutes in amounts greater than about 2 mg/ml.

In other aspects and embodiments, the invention provides a nicotine, (which includes nicotine polacrilex and nicotine salts, specifically salts such as nicotine benzoate, nicotine salicylate, nicotine succinate, nicotine pyruvate, nicotine citrate, and nicotine pyruvate) API composition. The composition comprises water, nicotine in amounts greater than about 6 mg/ml, and sugar in amounts greater than about 200 mg/ml, preferably from about 300 to about 700 mg/ml. Other embodiments of the composition comprise water, nicotine in amounts greater than about 6 mg/ml, and sugar substitutes in amounts greater than about 2 mg/ml, preferably from about 5 to about 20 mg/ml. Additional embodiments of the composition comprise water, nicotine in amounts greater than about 6 mg/ml, and sugar in amounts greater than about 200 mg/ml and sugar substitutes in amounts greater than about 2 mg/ml.

In other aspects and embodiments, the invention provides a composition having a combination of two or more APIs, for example a combination of the API codeine (also includes codeine phosphate) and the API guaifenesin. The composition comprises water, codeine in amounts greater than about 4 mg/ml, and guaifenesin in amounts greater than about 30 mg/ml, and sugar in amounts greater than about 200 mg/ml, preferably from about 300 to about 700 mg/ml. Other embodiments of the composition comprise water, codeine in amounts greater than about 4 mg/ml, and guaifenesin in amounts greater than about 30 mg/ml, and sugar substitutes in amounts greater than about 2 mg/ml, preferably from about 5 to about 20 mg/ml. Additional embodiments of the composition comprise water, codeine in amounts greater than about 4 mg/ml, and guaifenesin in amounts greater than about 30 mg/ml, and sugar in amounts greater than about 200 mg/ml and sugar substitutes in amounts greater than about 2 mg/ml.

In other aspects and embodiments, the invention provides a composition having a combination of four or more APIs, for example a combination of the API acetaminophen, dextromethorphan HBr, the API doxylamine succinate, and the API phenylephrine HCl. The composition comprises water, acetaminophen in amounts greater than about 33 mg/ml, dextromethorphan HBr in amounts greater than about 1 mg/ml, the API doxylamine succinate in amounts greater than about 0.6 mg/ml, and phenylephrine HCl in amounts greater than about 0.5 mg/ml, and sugar in amounts greater than about 200 mg/ml, preferably from about 300 to about 700 mg/ml. Other embodiments of the composition comprise water, acetaminophen in amounts greater than about 33 mg/ml, dextromethorphan HBr in amounts greater than about 1 mg/ml, the API doxylamine succinate in amounts greater than about 0.6 mg/ml, and phenylephrine HCl in amounts greater than about 0.5 mg/ml, and sugar substitutes in amounts greater than about 2 mg/ml, preferably from about 5 to about 20 mg/ml. Additional embodiments of the composition comprise water, acetaminophen in amounts greater than about 33 mg/ml, dextromethorphan HBr in amounts greater than about 1 mg/ml, the API doxylamine succinate in amounts greater than about 0.6 mg/ml, and phenylephrine HCl in amounts greater than about 0.5 mg/ml, and sugar in amounts greater than about 200 mg/ml and sugar substitutes in amounts greater than about 2 mg/ml.

In other aspects and embodiments, the invention provides a composition having a combination of four or more APIs, for example a combination of the API acetaminophen, the API dextromethorphan HBr, guaifenesin, and the API phenylephrine HC. The composition comprises water, acetaminophen in amounts greater than about 20 mg/ml, dextromethorphan HBr in amounts greater than about 1 mg/ml, guaifenesin in amounts greater than about 30 mg/ml, and phenylephrine HCl in amounts greater than about 0.5 mg/ml, and sugar in amounts greater than about 200 mg/ml, preferably from about 300 to about 700 mg/ml. Other embodiments of the composition comprise water, acetaminophen in amounts greater than about 20 mg/ml, dextromethorphan HBr in amounts greater than about 1 mg/ml, guaifenesin in amounts greater than about 30 mg/ml, and phenylephrine HCl in amounts greater than about 0.5 mg/ml, and sugar substitutes in amounts greater than about 2 mg/ml, preferably from about 5 to about 20 mg/ml. Additional embodiments of the composition comprise water, acetaminophen in amounts greater than about 20 mg/ml, dextromethorphan HBr in amounts greater than about 1 mg/ml, guaifenesin in amounts greater than about 30 mg/ml, and phenylephrine HCl in amounts greater than about 0.5 mg/ml, and sugar in amounts greater than about 200 mg/ml and sugar substitutes in amounts greater than about 2 mg/ml.

In other aspects and embodiments, the invention provides a composition having a combination of two or more APIs, for example a combination of the API dextromethorphan HBr and the API guaifenesin. The composition comprises water, dextromethorphan HBr in amounts greater than about 7 mg/ml, and guaifenesin in amounts greater than about 100 mg/ml, and sugar in amounts greater than about 200 mg/ml, preferably from about 300 to about 700 mg/ml. Other embodiments of the composition comprise water, dextromethorphan HBr in amounts greater than about 7 mg/ml, and guaifenesin in amounts greater than about 100 mg/ml, and sugar substitutes in amounts greater than about 2 mg/ml, preferably from about 5 to about 20 mg/ml. Additional embodiments of the composition comprise water, dextromethorphan HBr in amounts greater than about 7 mg/ml, and guaifenesin in amounts greater than about 100 mg/ml, and sugar in amounts greater than about 200 mg/ml and sugar substitutes in amounts greater than about 2 mg/ml.

Still other embodiments provide such API compositions where the API compositions comprise the encapsulating agent propylene glycol in amounts greater than about 90 mg/ml, preferably from about 90 to about 200 mg/ml. Yet other embodiments provide such API compositions where the API compositions comprise one or more flavorings. Further embodiments provide such API compositions where the API composition comprises a sugar, a sugar substitute, and a flavoring. Further embodiments provide such API compositions where the API composition comprises a sugar, a sugar substitute, and an encapsulating agent.

Yet other embodiments provide such API compositions where the API compositions comprise a buffer in amounts sufficient to provide the composition with a pH of between about 2 and about 7, where the composition has a pH of greater than about 2, or where the composition has a pH of less than about 6. Other embodiments provide such API compositions where the packaged composition has a shelf life of at least 3 months, at least 6 months, at least 12 months, at least 24 months, or at least 36 months.

Further embodiments provide multi-dose packages of API compositions. The packaged compositions comprise a bottle containing about 75 ml or less of the API composition. The API composition provides at least two doses of the API ingredient. Other embodiments provide such packaged compositions where the API composition provides at least about 10 doses.

Still other embodiments provide multi-dose packages of API compositions. The packaged composition comprises a bottle, a cap, a dosage cup, and shrink wrap. The bottle has a capacity of about 100 ml or less. At least two doses of the API composition are carried within the bottle. The dosage cup is releasably carried on the cap. The shrink wrap substantially envelopes the bottle and the dosage cup.

Yet other embodiments provide such packaged compositions where the packaged composition has an expanded content label adhered to the shrink wrap and where the expanded content label extends substantially all the way around a vertical wall of the bottle.

Further embodiments provide such packaged compositions where the bottle has a capacity of about 75 ml or less or where the bottle has at least about 10 doses of the syrup.

In other aspects and embodiments, the invention provides a composition having the supplement melatonin. The composition comprises water, melatonin in amounts greater than about 5 mg/ml, and sugar in amounts greater than about 200 mg/ml, preferably from about 300 to about 700 mg/ml. Other embodiments of the composition comprise water, melatonin in amounts greater than about 5 mg/ml, and sugar substitutes in amounts greater than about 2 mg/ml, preferably from about 5 to about 20 mg/ml. Additional embodiments of the composition comprise water, melatonin in amounts greater than about 5 mg/ml, and sugar in amounts greater than about 200 mg/ml and sugar substitutes in amounts greater than about 2 mg/ml.

In other aspects and embodiments, the invention provides a composition having the supplement vitamin A (as vitamin A palmitate). The composition comprises water, vitamin A (as vitamin A palmitate) in amounts greater than about 1.5 mg/ml, and sugar in amounts greater than about 200 mg/ml, preferably from about 300 to about 700 mg/ml. Other embodiments of the composition comprise water, vitamin A (as vitamin A palmitate) in amounts greater than about 1.5 mg/ml, and sugar substitutes in amounts greater than about 2 mg/ml, preferably from about 5 to about 20 mg/ml. Additional embodiments of the composition comprise water, vitamin A (as vitamin A palmitate) in amounts greater than about 1.5 mg/ml, and sugar in amounts greater than about 200 mg/ml and sugar substitutes in amounts greater than about 2 mg/ml.

In other aspects and embodiments, the invention provides a composition having the supplement B1 thiamin (as thiamin HCl or thiamine mononitrate). The composition comprises water, B1 thiamin (as thiamin HCl or thiamine mononitrate) in amounts greater than about 3 mg/ml, and sugar in amounts greater than about 200 mg/ml, preferably from about 300 to about 700 mg/ml. Other embodiments of the composition comprise water, B1 thiamin (as thiamin HCl or thiamine mononitrate) in amounts greater than about 3 mg/ml, and sugar substitutes in amounts greater than about 2 mg/ml, preferably from about 5 to about 20 mg/ml. Additional embodiments of the composition comprise water, B1 thiamin (as thiamin HCl or thiamine mononitrate) in amounts greater than about 3 mg/ml, and sugar in amounts greater than about 200 mg/ml and sugar substitutes in amounts greater than about 2 mg/ml.

In other aspects and embodiments, the invention provides a composition having the supplement B2 riboflavin (as riboflavin 5′ phosphate). The composition comprises water, B2 riboflavin (as riboflavin 5′ phosphate) in amounts greater than about 5 mg/ml, and sugar in amounts greater than about 200 mg/ml, preferably from about 300 to about 700 mg/ml. Other embodiments of the composition comprise water, B2 riboflavin (as riboflavin 5'phosphate) in amounts greater than about 5 mg/ml, and sugar substitutes in amounts greater than about 2 mg/ml, preferably from about 5 to about 20 mg/ml. Additional embodiments of the composition comprise water, B2 riboflavin (as riboflavin 5′ phosphate) in amounts greater than about 5 mg/ml, and sugar in amounts greater than about 200 mg/ml and sugar substitutes in amounts greater than about 2 mg/ml.

In other aspects and embodiments, the invention provides a composition having the supplement B3 (as niacin, niacinamide). The composition comprises water, B3 (as niacin, niacinamide) in amounts greater than about 75 mg/ml, and sugar in amounts greater than about 200 mg/ml, preferably from about 300 to about 700 mg/ml. Other embodiments of the composition comprise water, B3 (as niacin, niacinamide) in amounts greater than about 75 mg/ml, and sugar substitutes in amounts greater than about 2 mg/ml, preferably from about 5 to about 20 mg/ml. Additional embodiments of the composition comprise water, B3 (as niacin, niacinamide) in amounts greater than about 75 mg/ml, and sugar in amounts greater than about 200 mg/ml and sugar substitutes in amounts greater than about 2 mg/ml.

In other aspects and embodiments, the invention provides a composition having the supplement B5 (as pantothenic acid, d-calcium pantothenate). The composition comprises water, B5 (as pantothenic acid, d-calcium pantothenate) in amounts greater than about 20 mg/ml, and sugar in amounts greater than about 200 mg/ml, preferably from about 300 to about 700 mg/ml. Other embodiments of the composition comprise water, B5 (as pantothenic acid, d-calcium pantothenate) in amounts greater than about 20 mg/ml, and sugar substitutes in amounts greater than about 2 mg/ml, preferably from about 5 to about 20 mg/ml. Additional embodiments of the composition comprise water, B5 (as pantothenic acid, d-calcium pantothenate) in amounts greater than about 20 mg/ml, and sugar in amounts greater than about 200 mg/ml and sugar substitutes in amounts greater than about 2 mg/ml.

In other aspects and embodiments, the invention provides a composition having the supplement B6 (as pyridoxine hydrochloride). The composition comprises water, B6 (as pyridoxine hydrochloride) in amounts greater than about 75 mg/ml, and sugar in amounts greater than about 200 mg/ml, preferably from about 300 to about 700 mg/ml. Other embodiments of the composition comprise water, B6 (as pyridoxine hydrochloride) in amounts greater than about 75 mg/ml, and sugar substitutes in amounts greater than about 2 mg/ml, preferably from about 5 to about 20 mg/ml. Additional embodiments of the composition comprise water, B6 (as pyridoxine hydrochloride) in amounts greater than about 75 mg/ml, and sugar in amounts greater than about 200 mg/ml and sugar substitutes in amounts greater than about 2 mg/ml.

In other aspects and embodiments, the invention provides a composition having the supplement B7 (as d-biotin). The composition comprises water, B7 (as d-biotin) in amounts greater than about 5 mg/ml, and sugar in amounts greater than about 200 mg/ml, preferably from about 300 to about 700 mg/ml. Other embodiments of the composition comprise water, B7 (as d-biotin) in amounts greater than about 50 mg/ml, and sugar substitutes in amounts greater than about 2 mg/ml, preferably from about 5 to about 20 mg/m. Additional embodiments of the composition comprise water, B7 (as d-biotin) in amounts greater than about 50 mg/ml, and sugar in amounts greater than about 200 mg/ml and sugar substitutes in amounts greater than about 2 mg/ml.

In other aspects and embodiments, the invention provides a composition having the supplement B12 (as cyanocobalamin or methylcobalamin or adenosylcobalamin, or combination thereof). The composition comprises water, B12 (as cyanocobalamin or methylcobalamin or adenosylcobalamin, or combination thereof) in amounts greater than about 5 mg/ml, and sugar in amounts greater than about 200 mg/ml, preferably from about 300 to about 700 mg/ml. Other embodiments of the composition comprise water, B12 (as cyanocobalamin or methylcobalamin or adenosylcobalamin, or combination thereof) in amounts greater than about 5 mg/ml, and sugar substitutes in amounts greater than about 2 mg/ml, preferably from about 5 to about 20 mg/ml. Additional embodiments of the composition comprise water, B12 (as cyanocobalamin or methylcobalamin or adenosylcobalamin, or combination thereof) in amounts greater than about 5 mg/ml, and sugar in amounts greater than about 200 mg/ml and sugar substitutes in amounts greater than about 2 mg/ml.

In other aspects and embodiments, the invention provides for a composition having the combination of two or more supplements, for example a combination of six supplements, B1 thiamin (as thiamin HCl or thiamine mononitrate), B2 riboflavin (as riboflavin 5′ phosphate), B3 (as niacin, niacinamide), B5 (as pantothenic acid, d-calcium pantothenate), B6 (as pyridoxine hydrochloride), B12 (as cyanocobalamin or methylcobalamin or adenosylcobalamin, or combination thereof) composition. The six supplement composition comprises water, B1 thiamin (as thiamin HCl or thiamine mononitrate) in amounts greater than about 2 mg/ml, B2 riboflavin (as riboflavin 5′ phosphate) in amounts greater than about 2 mg/ml, B3 (as niacin, niacinamide) in amounts greater than about 5 mg/ml, B5 (as pantothenic acid, d-calcium pantothenate) in amounts greater than about 5 mg/ml, B6 (as pyridoxine hydrochloride) in amounts greater than about 2 mg/ml, B12 (as cyanocobalamin or methylcobalamin or adenosylcobalamin, or combination thereof) in amounts greater than about 2.5 mg/ml, and sugar in amounts greater than about 200 mg/ml, preferably from about 300 to about 700 mg/ml. The six supplement composition comprises water, B1 thiamin (as thiamin HCl or thiamine mononitrate) in amounts greater than about 2 mg/ml, B2 riboflavin (as riboflavin 5′ phosphate) in amounts greater than about 2 mg/ml, B3 (as niacin, niacinamide) in amounts greater than about 5 mg/ml, B5 (as pantothenic acid, d-calcium pantothenate) in amounts greater than about 5 mg/ml, B6 (as pyridoxine hydrochloride) in amounts greater than about 2 mg/ml, B12 (as cyanocobalamin or methylcobalamin or adenosylcobalamin, or combination thereof) in amounts greater than about 2.5 mg/ml, and sugar substitutes in amounts greater than about 2 mg/ml, preferably from about 5 to about 20 mg/ml. The six supplement composition comprises water, B1 thiamin (as thiamin HCl or thiamine mononitrate) in amounts greater than about 2 mg/ml, B2 riboflavin (as riboflavin 5′ phosphate) in amounts greater than about 2 mg/ml, B3 (as niacin, niacinamide) in amounts greater than about 5 mg/ml, B5 (as pantothenic acid, d-calcium pantothenate) in amounts greater than about 5 mg/ml, B6 (as pyridoxine hydrochloride) in amounts greater than about 2 mg/ml, B12 (as cyanocobalamin or methylcobalamin or adenosylcobalamin, or combination thereof) in amounts greater than about 2.5 mg/ml, and sugar in amounts greater than about 200 mg/ml and sugar substitutes in amounts greater than about 2 mg/ml.

In other aspects and embodiments, the invention provides a composition having the supplement vitamin D (as cholecalciferol). The composition comprises water, vitamin D (as cholecalciferol) in amounts greater than about 0.5 mg/ml, and sugar in amounts greater than about 200 mg/ml, preferably from about 300 to about 700 mg/ml. Other embodiments of the composition comprise water, vitamin D (as cholecalciferol) in amounts greater than about 0.5 mg/ml, and sugar substitutes in amounts greater than about 2 mg/ml, preferably from about 5 to about 20 mg/ml. Additional embodiments of the composition comprise water, vitamin D (as cholecalciferol) in amounts greater than about 0.5 mg/ml, and sugar in amounts greater than about 200 mg/ml and sugar substitutes in amounts greater than about 2 mg/ml.

In other aspects and embodiments, the invention provides a composition having the supplement vitamin E (as dl-alpha tocopheryl acetate). The composition comprises water, vitamin E (as dl-alpha tocopheryl acetate) in amounts greater than about 50 mg/ml, and sugar in amounts greater than about 200 mg/ml, preferably from about 300 to about 700 mg/ml. Other embodiments of the composition comprise water, vitamin E (as dl-alpha tocopheryl acetate) in amounts greater than about 50 mg/ml, and sugar substitutes in amounts greater than about 2 mg/ml, preferably from about 5 to about 20 mg/ml. Additional embodiments of the composition comprise water, vitamin E (as dl-alpha tocopheryl acetate) in amounts greater than about 50 mg/ml, and sugar in amounts greater than about 200 mg/ml and sugar substitutes in amounts greater than about 2 mg/ml.

In other aspects and embodiments, the invention provides a composition having the supplement curcumin tumeric. The composition comprises water, curcumin tumeric in amounts greater than about 150 mg/ml, and sugar in amounts greater than about 200 mg/ml, preferably from about 300 to about 700 mg/ml. Other embodiments of the composition comprise water, curcumin tumeric in amounts greater than about 150 mg/ml, and sugar substitutes in amounts greater than about 2 mg/ml, preferably from about 5 to about 20 mg/ml. Additional embodiments of the composition comprise water, curcumin tumeric in amounts greater than about 150 mg/ml, and sugar in amounts greater than about 200 mg/ml and sugar substitutes in amounts greater than about 2 mg/ml.

Still other embodiments provide such supplement compositions where the supplement compositions comprise the encapsulating agent propylene glycol in amounts greater than about 90 mg/ml, preferably from about 90 to about 200 mg/ml. Yet other embodiments provide such supplement compositions where the supplement compositions comprise one or more flavorings. Further embodiments provide such supplement compositions where the supplement composition comprises a sugar, a sugar substitute, and a flavoring. Further embodiments provide such supplement compositions where the supplement composition comprises a sugar, a sugar substitute, and an encapsulating agent.

Yet other embodiments provide such supplement compositions where the supplement compositions comprise a buffer in amounts sufficient to provide the composition with a pH of between about 2 and about 7, where the composition has a pH of greater than about 2, or where the composition has a pH of less than about 6. Other embodiments provide such supplement compositions where the packaged composition has a shelf life of at least 3 months, at least 6 months, at least 12 months, at least 24 months, or at least 36 months.

Further embodiments provide multi-dose packages of supplement compositions. The packaged compositions comprise a bottle containing about 75 ml or less of the supplement composition. The supplement composition provides at least two doses of the dietary supplement ingredient. Other embodiments provide such packaged compositions where the supplement composition provides at least about 10 doses.

Still other embodiments provide multi-dose packages of supplement compositions. The packaged composition comprises a bottle, a cap, a dosage cup, and shrink wrap. The bottle has a capacity of about 100 ml or less. At least two doses of the supplement composition are carried within the bottle. The dosage cup is releasably carried on the cap. The shrink wrap substantially envelopes the bottle and the dosage cup.

Yet other embodiments provide such packaged compositions where the packaged composition has an expanded content label adhered to the shrink wrap and where the expanded content label extends substantially all the way around a vertical wall of the bottle.

Further embodiments provide such packaged compositions where the bottle has a capacity of about 75 ml or less or where the bottle has at least about 10 doses of the syrup.

Finally, still other aspects and embodiments of the invention will have various combinations of such features as will be apparent to workers in the art.

Thus, the present invention in its various aspects and embodiments comprises a combination of features and characteristics that are directed to overcoming various shortcomings of the prior art. The various features and characteristics described above, as well as other features and characteristics, will be readily apparent to those skilled in the art upon reading the following detailed description of the preferred embodiments and by reference to the appended drawings.

Moreover, the contents of this patent application are presented solely for the purpose of being reviewed by the United States Patent and Trademark Office or its counterparts in other countries for patentability of the claimed novel OTC drug formulations and packaged formulations. In accordance with the Federal Food, Drug, and Cosmetic Act of 1938 (FD&C), the Nutrition Labeling and Education Act of 1990 (NLEA), and the Dietary Supplement Health and Education Act of 1994 (DSHEA), it will be understood that statements made within this patent application or before other administrative agencies have not been evaluated by the FDA. Further in accordance with FD&C, NLEA, and the DSHEA, applicant is not asserting that any formulations disclosed herein are intended to diagnose, treat, prevent, mitigate or cure disease.

### BRIEF DESCRIPTION OF THE DRAWINGS

FIG. 1 is an isometric view of a first preferred embodiment 10 of the novel multi-dose packaged OTC liquid formulations, which packaged formulation 10 has a shrink wrap inner label 15 and a folded outer label 16.

FIG. 2 is an elevational view of packaged formulation 10 shown in FIG. 1.

FIG. 3 is an exploded view of packaged formulation 10 showing the various components thereof.

FIG. 4 is a plan view of unwrapped inner shrink wrap 15 of packaged formulation 10.

FIG. 5A is a plan view of unrolled folded outer label 16 of packaged formulation 10 showing the outer fold thereof.

FIG. 5B is a plan view of unfolded outer label 16 of packaged formulation 10 showing the inner fold thereof.

In the drawings and description that follows, like parts are identified by the same reference numerals. The drawing figures are not necessarily to scale. Certain features of the embodiments may be shown exaggerated in scale or in somewhat schematic form and some details of conventional design and construction may not be shown in the interest of clarity and conciseness.

### DESCRIPTION OF ILLUSTRATIVE EMBODIMENTS

### Overview of Novel OTC Formulations

The novel OTC formulations are liquid formulations, often referred to as syrups. They are intended to be taken orally, and thus the base fluid is water. In general, they comprise one or more active pharmaceutical ingredients and one or more excipients. Active pharmaceutical ingredients (“APIs”) are the compounds that make a drug formulation effective. They act pharmacologically to relieve symptoms or cure an underlying medical condition.

Excipients, also known as inactive ingredients, are pharmacologically inert substances that aid in delivery of the active ingredients. For example, excipients may be used to give a formulation its form, such as using cornstarch to make a tablet or sterile water to make a syrup. Other excipients may be used to control the release of the active ingredient once it is ingested. Excipients also may be used to maintain the stability of the formulation or to improve the taste or appearance of the formulation.

An OTC drug formulation, including its active ingredients and excipients, must meet various regulatory requirements in virtually all, if not all countries in order to be marketed and sold. In the United States, such regulations fall largely within 21 C.F.R. §§ 330 et seq. (Over-the Counter (OTC) Human Drugs Which are Generally Recognized as Safe and Effective and not Misbranded), and the Tentative and Final Monographs included therein. For example, the Final Monograph for DXM HBr and other oral antitussive drugs may be found at 21 C.F.R. §§ 341 et seq. Responsibility for promulgating and administering such regulations falls within the purview of the United States Food and Drug Administration (FDA). The FDA also maintains a list of approved excipients and the forms and amounts in which they may be used. In the European Union, the European Medicines Agency (EMA) assumes a similar role, and in Japan it is the Pharmaceuticals and Medical Devices Agency (PMDA).

As used herein, Regulatory Guidelines shall be understood to refer to and include the statutes and regulations governing the marketing and sale of over-the-counter drugs, including, but not limited to the allowed dosages of active ingredients for a particular indication. Regulatory Agencies shall be understood to include the FDA, EMA, PMDA, and equivalent agencies in other countries.

As used herein, “stability” refers to the ability of an OTC formulation to resist aggregation, fragmentation, deamidation, oxidation, or other forms of chemical and physical degradation that affect its pharmacological activity or the acceptability of its taste.

As used herein, a “stable” formulation is a packaged formulation that meets Regulatory Guidelines, including those relating to identity, strength, quality, and purity, over a specified period of time.

As used herein, “shelf life” of a packaged formulation is the period of time over which a formulation is “stable.”

### Active OTC Ingredients

The active ingredient in the novel OTC drug formulations may be any active ingredient approved for use in liquid drug formulations to be sold directly to consumers under US Regulatory Guidelines or Regulatory Guidelines of another country. Preferred, active ingredients may include those listed in the FDA Final Monographs for antacids (21 C.F.R. § 331), antiflatulents (antigas) (21 C.F.R. § 332), antidiarrheal (21 C.F.R. § 335), antiemetic (21 C.F.R. § 336), nighttime sleep-aids (21 C.F.R. § 338), stimulants (21 C.F.R. § 340), cold, cough (antitussive), allergy, bronchodilator, expectorant, nasal decongestant, and antiasthmatic (21 C.F.R. § 341), analgesic-antipyretic, cardiovascular, rheumatologic (21 C.F.R. § 343).

As described further below, the active ingredient will be present in concentrated amounts allowing for the packaging of multiple doses in a small bottle. For example, DXM HBr may be added in amounts from about 6 to about 25 milligrams per milliliter (mg/ml) of formulation, preferably from about 7 to about 10 mg/ml. As further examples, the novel OTC formulations may comprise dextromethorphan HBr in amounts greater than about 7 mg/ml, dextromethorphan polistirex in amounts greater than about 9 mg/ml, acetaminophen in amounts greater than about 50 mg/ml, aluminum hydroxide in amounts greater than about 120 mg/ml, bismuth subsalicylate in amounts greater than about 26 mg/ml, caffeine in amounts greater than about 6 mg/ml, centirizine HCl in amounts greater than about 1.5 mg/ml, choline salicylate in amounts greater than about 23 mg/ml, one or more over-the-counter active pharmaceutical codeine ingredients selected from the group consisting of codeine or codeine phosphate, the ingredients being present in total amounts greater than about 7 mg/ml, diphenhydramine HCl in amounts greater than about 4 mg/ml, doxylamine succinate in amounts greater than about 1.5 mg/ml, fexofenadine HCl in amounts greater than about 9 mg/ml, guaifenesin in amounts greater than about 45 mg/ml, ibuprofen in amounts greater than about 60 mg/ml, loratadine in amounts greater than about 1.5 mg/ml, magnesium hydroxide in amounts greater than about 120 mg/ml, phenylephrine HCl in amounts greater than about 1 mg/ml, simethicone in amounts greater than about 100 mg/ml, aspirin in amounts greater than about 750 mg/ml, bisacodyl in amounts greater than about 15 mg/ml, calcium carbonate in amounts greater than about 3,000 mg/ml, cimetidine in amounts greater than about 300 mg/ml, dimenhydrinate in amounts greater than about 38 mg/ml, esomeprazole magnesium in amounts greater than about 30 mg/ml, famotidine in amounts greater than about 30 mg/ml, levocetirizine dihydrochloride in amounts greater than about 7.5 mg/ml, loperamide HCl in amounts greater than about 6 mg/ml, naproxen sodium in amounts greater than about 330 mg/ml, omeprazole in amounts greater than about 30 mg/ml, ranitidine HCl in amounts greater than about 250 mg/ml, and one or more over-the-counter nicotine ingredients selected from the group consisting of nicotine polacrilex and nicotine salts, the nicotine salts being selected from the group consisting of nicotine benzoate, nicotine salicylate, nicotine succinate, nicotine pyruvate, nicotine citrate, and nicotine pyruvate, the nicotine ingredients being present in total amounts greater than about 6 mg/ml.

In other embodiments, the formulation will approach saturation when fully formulated so as to minimize the volume of formulation required to deliver a single dose of active ingredient while still passing required testing. As discussed further below, the novel, high concentration API formulations will allow multiple doses of liquid formulation to be distributed in much smaller packaging.

It will be appreciated that the novel formulations may include more than one API in a single formulation. For example, a multi-symptom cold/flu formulation may contain four APIs, acetaminophen, dextromethorphan HBr, guaifenesin, and phenylephrine HCl to provide more relief of multiple ailments. They also may include other types of APIs. Preferred combinations will be those listed in the FDA Final Monographs.

### Preferred Excipients

Preferred embodiments of the novel OTC liquid formulations have high concentrations of APIs and other inactive ingredients. Thus, encapsulating agents preferably will be used to facilitate dissolution of concentrated APIs and other inactive ingredients in embodiments of the novel formulations. Sweet tasting encapsulating agents, such as propylene glycol, are preferred.

APIs and other inactive ingredients are known to have bitter taste that may discourage consumers from following recommended dosages at recommended intervals. That bitterness is exacerbated by increasing the concentration of APIs. Thus, the novel OTC compositions having high concentrations of APIs preferably include high concentrations of sugar, and/or sugar substitutes.

Sugar and sugar substitutes are believed to provide both an antienemic effect and a taste masking effect. One or more sugars may be included in the formulation. Suitable sugars may include, but are not limited to, glucose, dextrose, disaccharides, fructose (aka levulose), galactose, high fructose corn syrup, lactose, maltose, trisaccharides, and sucrose. One or more sugar substitutes may be included in addition to or as a substitute for sugars. Sugars may be added in concentrations greater than about 200 mg/ml, preferably from about 400 to about 700 mg/ml.

Suitable sugar substitutes may include, but are not limited to, acesulfame potassium, advantame, alitame, aspartame, brazzein, curculin, dulcin, erythritol, fructooligosaccharide, glucin, glycerol, glycyrrhizin, hydrogenated starch hydrolysates, inulin, isomalt, isomaltooligosaccharide, isomaltulose, lactitol, mabinlin, maltitol, maltodextrin, mannitol, miraculin, mogroside mix, monatin, monellin, neohesperidin dihydrochalcone, neotame, osladin, pentadin, polydextrose, psicose, saccharin, salt of aspartame-acesulfame, sodium cyclamate, sorbitol, stevia, sucralose, tagatose, thaumatin, and xylitol. Sugar substitutes may be added in concentrations greater than about 2 mg/ml, preferably from about 5 to about 20 mg/ml.

One or more flavorings also may be included to mask the taste of APIs. Suitable flavorings may include, but are not limited to, artificial flavors, artificial vanilla, dimethyl anthranilate, eculyptol, menthol, methyl anthranilate, methyl salicylate, natural flavors, peppermint, thymol, various fruit flavors, and culinary herbs and spices. Flavorings typically will be added for taste.

Other preferred embodiments may comprise buffers to adjust the pH of the formulation. Suitable buffering agents will be minimally reactive with other components of the formulation and may be more acidic or more basic. Typically, buffering agents will be present in the range of from 0 to about 1 g/ml of the formulation.

For example, buffering agents may be included in amounts sufficient to provide the formulation with a pH of between about 2 and about 7, where the formulation has a pH of greater than about 2, or where the formulation has a pH of less than about 6. In certain formulations, it has been found that adjusting the pH to certain levels can allow a the novel high-concentrated formulation to have a longer shelf life, such as a shelf life of at least 3 months, at least 6 months, at least 12 months, at least 24 months, or at least 36 months.

### Other Excipients

Preferred embodiments also may include other excipients, such as extenders, diluents, wetting agents, solvents, emulsifiers, preservatives, absorption enhancers, sustained-release matrices, and coloring agents. Preferred excipients will be those listed for use in OTC drug formulations.

### Overview of Novel Dietary Supplement Formulations

The novel dietary supplement formulations also are liquid formulations or syrups. As with the novel OTC formulations, they are intended to be taken orally, and thus the base fluid is water. In general, they comprise one or more active ingredients intended to supplement the diet of humans and one or more pharmacologically inert excipients.

### Active Dietary Supplement Ingredients

The active ingredient in the novel dietary supplement formulations may be any dietary supplement ingredient for use in liquid formulations sold directly to consumers under US Regulatory Guidelines or Regulatory Guidelines of another country. The dietary ingredients include vitamins, minerals, herb, or other botanicals, amino acids, and other dietary substances for use by man to supplement diets by increasing the total dietary intake, as well as concentrates, metabolites, constituents, extracts, or combinations of the such substances.

Preferred dietary ingredients include melatonin, vitamin A (as vitamin A palmitate), B1 vitamins selected from the group consisting of thiamin HCl and thiamine mononitrate, vitamin B2 riboflavin (as riboflavin 5′ phosphate), one or both B3 vitamins selected from the group consisting of niacin and niacinamide, one or both B5 vitamins selected from the group consisting of pantothenic acid and d-calcium pantothenate, vitamin B6 (as pyridoxine hydrochloride), vitamin B7 (as d-biotin), one or more B12 vitamins selected from the group consisting of cyanocobalamin, methylcobalamin, and adenosylcobalamin, vitamin D (as cholecalciferol), vitamin E (as di-alpha tocopheryl acetate), and curcumin turmeric.

As described further below, the dietary ingredient will be present in concentrated amounts allowing for the packaging of multiple doses in a small bottle. For example, melatonin may be present in amounts greater than about 5 mg/ml, vitamin A (as vitamin A palmitate) may be present in amounts greater than about 1.5 mg/ml, B1 vitamins selected from the group consisting of thiamin HCl and thiamine mononitrate may be present in total amounts greater than about 3 mg/ml, vitamin B2 riboflavin (as riboflavin 5′ phosphate) may be present in amounts greater than about 5 mg/ml, one or both B3 vitamins selected from the group consisting of niacin and niacinamide may be present in total amounts greater than about 75 mg/ml, one or both B5 vitamins selected from the group consisting of pantothenic acid and d-calcium pantothenate may be present in total amounts greater than about 20 mg/ml, vitamin B6 (as pyridoxine hydrochloride) may be present in amounts greater than about 75 mg/ml, vitamin B7 (as d-biotin) may be present in amounts greater than about 5 mg/ml, one or more B12 vitamins selected from the group consisting of cyanocobalamin, methylcobalamin, and adenosylcobalamin may be present in total amounts greater than about 5 mg/ml, vitamin D (as cholecalciferol) may be present in amounts greater than about 0.5 mg/ml, vitamin E (as dl-alpha tocopheryl acetate) may be present in amounts greater than about 50 mg/ml, and curcumin tumeric may be present in amounts greater than about 150 mg/ml.

In other embodiments, the dietary ingredient will approach saturation when fully formulated so as to minimize the volume of formulation required to deliver a single dose of dietary ingredient while still passing required testing. As discussed further below, the novel, high concentration dietary supplement formulations will allow multiple doses of liquid formulation to be distributed in much smaller packaging.

It will be appreciated that the novel dietary supplement formulations may include more than one dietary ingredient in a single formulation. For example, a package of B vitamins may be provided in a single formulation. Such formulations may include, for example, two or more B vitamins selected from the group consisting of one or both B1 vitamins selected from the group consisting of thiamin HCl and thiamine mononitrate, vitamin B2 riboflavin (as riboflavin 5′ phosphate), one or both B3 vitamins selected from the group consisting of niacin and niacinamide, one or both B5 vitamins selected from the group consisting of pantothenic acid and d-calcium pantothenate, vitamin B6 (as pyridoxine hydrochloride), vitamin B7 (as d-biotin), and one or more B12 vitamins selected from the group consisting of cyanocobalamin, methylcobalamin, and adenosylcobalamin.

### Excipients

Preferred embodiments of the novel liquid dietary supplement formulations have high concentrations of dietary ingredients and other inactive ingredients. Thus, encapsulating agents preferably will be used to facilitate dissolution of concentrated dietary ingredients and other inactive ingredients in embodiments of the novel formulations. Sweet tasting encapsulating agents, such as propylene glycol, are preferred.

Some dietary ingredients and other inactive ingredients are known to have bitter taste that may discourage consumers from following recommended dosages at recommended intervals. That bitterness is exacerbated by increasing the concentration of dietary ingredients. Thus, the novel dietary supplement formulations having high concentrations of dietary ingredients preferably include high concentrations of sugar, and/or sugar substitutes as present in the novel OTC liquid formulations. The sugars and sugar substitutes, and the concentrations thereof, described above as generally suited for use in the novel OTC liquid formulations also may be suitable for use in the novel dietary supplement formulations.

Likewise, the novel liquid dietary supplement formulations may include one or more flavorings as described above to mask the taste of dietary supplements. Buffers also may be used as described above, as may other excipients, such as extenders, diluents, wetting agents, solvents, emulsifiers, preservatives, absorption enhancers, sustained-release matrices, and coloring agents.

### Novel Multi-Dose Packaged OTC Formulations

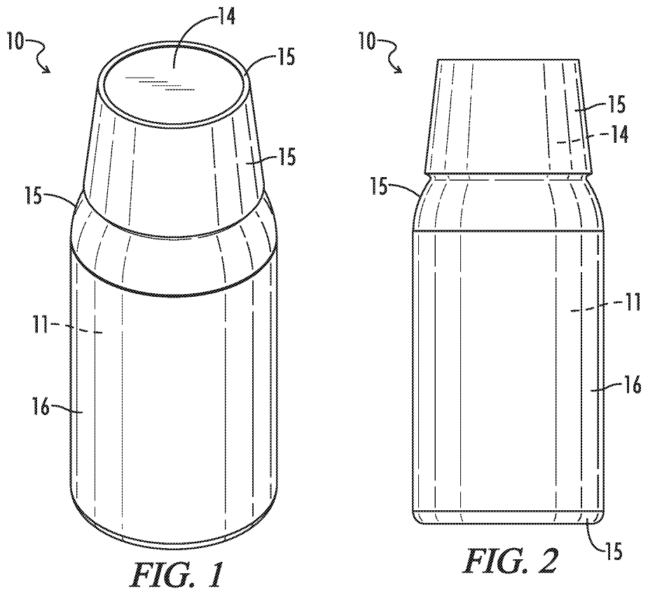

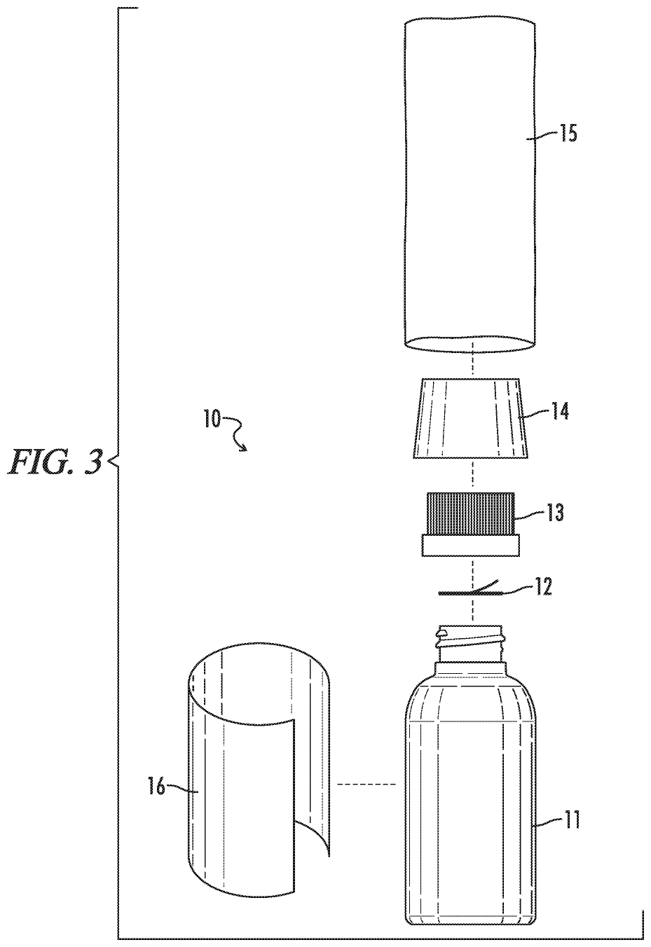

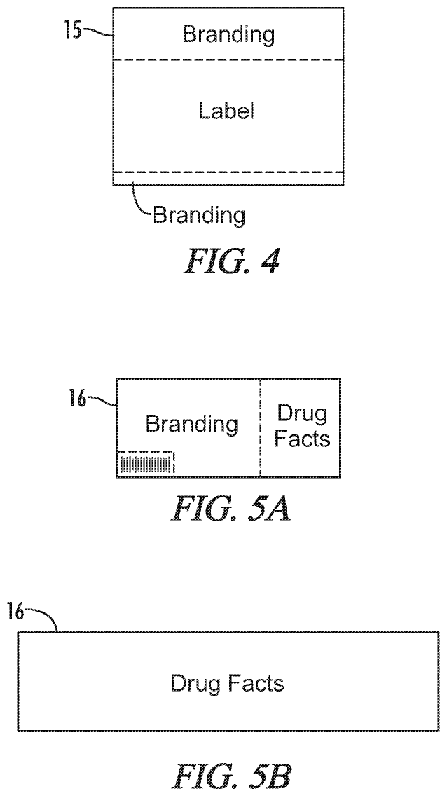

Preferred embodiments of the invention will provide multiple doses of a liquid OTC active ingredient or dietary supplement ingredient in a relatively small bottle. For example, a preferred multi-dose packaged OTC formulation 10 is shown in FIGS. 1-5. As shown therein, packaged formulation 10 generally includes a bottle 11, a seal 12, a cap 13, a dosage cup 14, an inner shrink wrap 15, and an outer expanded content label 16. Bottle 11 and cap 13 may be of any conventional design and shape suitable for holding liquids and many suitable bottles and caps are available commercially.

Bottle 11, for example, is a “Boston round” design. It has a generally cylindrical shape with relatively long vertical walls. The top of bottle 11 tapers rapidly into a relatively flat shoulder surrounding a relatively small neck and opening. Bottle 11 may be made from conventional material by conventional methods. Preferably, however, bottle 11 is made from polymers, such as polypropylene, high-density and other polyethylenes, and polyethylene terephthalate, by blow molding. As noted, the shape of bottle 11 and cap 13 is not critical. Importantly, however, bottle 11 is relatively small, preferably having a capacity of less than 100 ml, and more preferably, less than about 75 or 50 ml.

The opening of bottle 11 preferably is sealed with seal 12. Seal 12 not only helps to preserve the formulation, but it can provide an indicator that the product has not been tampered with or adulterated. Seal 12 may be fabricated from conventional materials and applied by conventional methods. For example, a ‘lift-n-peel” induction seal may be used and sealed over the opening of bottle 11 by induction heating. Lift-n-peel liners provide a tab by which a consumer can peel the seal off.

The neck of bottle 11 and cap 13 may incorporate any conventional design that allows cap 13 to provide a liquid-tight closure for bottle 11, such as a threaded cap. Preferably, however, cap 13 is a child resistant cap. Many conventional child resistant caps are known and may be used, such as a “push and turn” cap. Typically, such caps are molded from plastics such as polypropylene or polyethylene terephthalate and are widely available. Cap 13 also may incorporate a liner to aid in sealing the opening.

Dosage cup 14, as its name implies, is a small open cup that is primarily designed to allow a consumer to measure a recommended dose of the liquid formulation. Preferably it is fabricated from a clear plastic, such as polypropylene, and is embossed with markings indicating the recommended dosage line. A consumer may more accurately confirm that the amount of formulation in the cup matches the recommended dose.

The length of dosage cup 14 is not particularly critical so long as it provides sufficient depth to accommodate the volume of a dose. The inner diameter of dosage cup 14, however, is preferably sized and configured to fit securely over cap 13. Many conventional designs are known and may be used. For example, dosage cup 14 may be provided with interior vertical ribs. When dosage cup 14 is placed over cap 13 the ribs will provide a friction fit. Ribs also may be designed to clip on to the underside of cap 13. In any event, dosage cup 14 preferably is releasably secured to cap 13, minimizing the likelihood of it being misplaced or lost by a consumer, but ensuring that it is readily available for use.

Typically, bottle 11 will be filled to slightly less than capacity to avoid spillage during packaging. Thus, bottle 11 preferably will be filled to less than about 75 ml, and preferably less than about 50 ml or less than about 40 ml. Preferably, the amount of syrup contained in packaged formulation 10 will be coordinated with the concentration of DXM HBr to provide a specific number of recommended doses.

The FDA Final Monograph lists the dosages under which DXM HBr OTC drugs may be marketed. For adults and children 12 years of age or older the oral dosage is 10 to 20 mg every 4 hours or 30 mg every 6 to 8 hours, not to exceed 120 mg in 24 hours, or as directed by a doctor. For children 6 to under 12 years of age the oral dosage is 12.5 mg every 4 hours, not to exceed 75 mg in 24 hours, or as directed by a doctor. OTC formulations containing DXM HBr are not to be used for children under 6 years of age except as recommended by a doctor.

Thus, when present in amounts of about 10 mg/ml, for example, a single adult, 6 to 8-hour dose of the formulation will be 3 ml, and 15 doses will be 45 ml. Dosage cup 14 in this example would provide a 3 ml indicator line. If the formulation were labeled for recommended adult, 4-hour doses of 20 mg, dosage cup would provide a ml indicator line. Fifteen doses could be provided by 30 ml of formulation, or 30 doses could be provided by 60 ml of formulation. In general, therefore, the bottle will be filled with sufficient formulation to provide at least about 60 mg of DXM HBr, or at least about 120 mg of DXM HBr, or at least about 300 mg of DXM HBr, or at least about 450 mg of DXM HBr. The formulation will be packaged to provide at least 2 doses. More commonly, it may be provided with at least about 4 doses, or at least about 10 doses, or at least about 15 doses.

Shrink wrap 15 may be used to secure dosage cup 14 during packing, shipment, and sale. It also may provide an indication of tampering or adulteration. Shrink wrap 15 may be any suitable conventional shrink wrap, such as those made from polyvinyl chloride and polyolefins. A relatively narrow band may be provided and will be sufficient to secure dosage cup 14. Preferably, however, shrink wrap 15 will substantially envelope the assembly of bottle 11, cap 13, and dosage cup 14. For example, as may be seen in FIGS. 1-2, shrink wrap 15 extends from partially under the bottom of bottle 11 all the way up and partially over the bottom of dosage cup 14. Dosage cup 14 is secured to bottle 11 and cap 13, and as discussed further below, shrink wrap 15 can provide a substrate on which branding and other messages may be imprinted. Perforations or other weakened areas may be provided in shrink wrap 15 to allow a consumer to easily remove dosage cup 14. Preferably, especially if shrink wrap 15 is imprinted with branding or other messages, the perforations will allow dosage cup 14 to be removed while leaving most of shrink wrap 15 on bottle 11.

Expanded content label 16 is affixed to shrink wrap 15. Many conventional expanded content labels are available commercially and may be used. Such labels have multiple plies which provide a substrate on which branding and other messages may be imprinted. Label 16, for example, is a continuous web having a single lateral fold. The back of the label is provided with a relatively strong adhesive such that label 16 is self-adhering. The folded portions of label 16 are lightly adhered so that they may be peeled apart to expose their inner sides. Preferably, as shown in FIGS. 1-2, label 16, when folded and applied to bottle 11 extends substantially completely around bottle 11. Doing so will avoid the need to register label 16 with indicia imprinted on shrink wrap 15. Other types of expanded content labels may be used if desired. For example, other expanded content labels have one or more webs that are joined or folded to form a booklet.

It will be appreciated, therefore, that embodiments of the novel packaged OTC and dietary supplement formulations have significant advantages over the prior art. By packaging a highly concentrated liquid formulation in a relatively small bottle, the packaged formulation may contain multiple doses yet may be easily accommodated on a retailer's shelf. As noted previously, that is particularly critical for retailers, such as convenience stores, where shelf space in severely constrained.

Moreover, while other forms of labeling may be used if desired, by providing a combination of a full shrink wrap and an expanded content label, such as shrink wrap 15 and label 16, the packaging may be provided with sufficient space to comply with regulatory labeling requirements while allowing space for branding and other optional messages and bar coding. It may not be necessary to distribute individual products in additional packaging, such as individual paperboard boxes.

It will be appreciated that various functions and mechanisms are ascribed to each component of the novel formulations and packaged formulations and to their effect on the overall properties thereof. While such explanations are believed to be accurate, and are believed to provide useful guidance in making and using various embodiments of the novel formulations and packaged formulations, it will be understood that the invention is not limited thereby. The economics and characteristics of a particular embodiment also may render it more suitable for particular purposes. One embodiment may be well suited for one application and unsuited for another. Thus, general statements should be taken as such, and not as definitive, immutable principles.

### EXAMPLES

The invention and its advantages may be further understood by reference to the following examples. It will be appreciated, however, that the invention is not limited thereto.

### Example 1—Prior Art

A sample of a Robitussin Adult Peak Cold liquid product was purchased at a convenience store. The product was labeled as Cough+Chest Congestion DM. The syrup was packaged in a bottle containing 4 ounces (118 ml) of syrup. The syrup contained 2 mg/ml of DXM HBr and 20 mg/ml guaifenesin (an expectorant). The recommended adult dose was 20 mg, and thus the size of the dose was 10 ml. The bottle contained approximately 11 to 12 doses.

The bottle was packaged in a paperboard box measuring approximately 2″ wide, 2″ deep, and 5″ tall. It is estimated that a dozen such products will occupy 48 square inches of shelf space. Stacked 3-deep, a dozen products will require approximately 8″ of shelf frontage.

### Example 2

A novel liquid API composition was prepared having the components set forth in Table 1 below.

TABLE 1

| Component | Concentration (mg/mL) |
|---|---|
| Sodium Benzoate | 1 |
| Flavoring | 2 |
| Phosphoric acid | 4.9 |
| Sugar substitute | 8 |
| API | 10 |
| Propylene Glycol | 100 |
| Water | 435 |
| Sugar | 650 |

Sugar was added to a first tank containing a portion of the indicated water and heated to 40° C. Approximately half of the water was heated in a second tank to 40° C. The sodium benzoate and sugar substitute were added to Tank 2 and stirred until dissolved. The heated contents of Tank 2 were transferred to Tank 1. Tank 2 was rinsed twice, each rinse being approximately 5% of the indicated water, and the rinse added to Tank 1. Propylene glycol was added to a third tank and heated to 40° C. An API was added to the heated propylene glycol and stirred until dissolved. Flavoring was added to Tank 3 and stirred until dissolved. Phosphoric acid was added to Tank 3 and stirred until homogeneity. The contents of Tank 3 were added to Tank 1. Tank 3 was rinsed twice, each rinse being 21 approximately 5% of the indicated water. The remaining water was added to Tank 1 and the formulation was stirred to homogeneity.

The formulation was tasted and found to have an acceptable taste with no bitterness.

### Example 3

A novel liquid diphenhydramine HCl API composition may be prepared as set forth below in Table 2. A citric acid buffering agent was used to provide the composition with a pH of 5.5. It is expected that it will have an acceptable taste with no bitterness and a shelf life of over 12 months.

TABLE 2

| Component | Concentration (mg/mL) |
|---|---|
| Sodium Benzoate | 1 |
| Flavoring | 2 |
| Citric Acid | 4.9 |
| Sugar substitute | 8 |
| Diphenhydramine HCl | 30 |
| Propylene Glycol | 100 |
| Water | 435 |
| Sugar | 650 |

### Example 4

Other liquid API composition may be prepared as set forth below and it is expected that all will have an acceptable taste with no bitterness.

Composition No. 1, a composition comprising:
(a) water;
(b) dextromethorphan hydrobromide in amounts greater than about 7 mg/ml; and
(c) sugar in amounts greater than about 200 mg/ml; and (optionally)
(d) a buffering agent sufficient to achieve a pH of about 2.0 to 7.0.

Composition No. 2, a composition comprising:
(a) water;
(b) dextromethorphan hydrobromide in amounts greater than about 7 mg/ml; and
(c) sugar substitutes in amounts greater than about 2 mg/ml; and (optionally)
(d) a buffering agent sufficient to achieve a pH of about 2.0 to 7.0.

Composition No. 3, a composition comprising:
(a) water;
(b) dextromethorphan hydrobromide in amounts greater than about 7 mg/ml; and
(c) sugar in amounts greater than about 200 mg/ml; and
(d) sugar substitutes in amounts greater than about 2 mg/ml.

Composition No. 4, a composition comprising:
(a) water;
(b) dextromethorphan polistirex in amounts greater than about 9 mg/ml; and
(c) sugar in amounts greater than about 200 mg/ml.

Composition No. 5, a composition comprising:
(a) water;
(b) dextromethorphan polistirex in amounts greater than about 9 mg/ml; and
(c) sugar substitutes in amounts greater than about 2 mg/ml.

Composition No. 6, a composition comprising:
(a) water;
(b) dextromethorphan polistirex in amounts greater than about 9 mg/ml; and
(c) sugar in amounts greater than about 200 mg/ml; and
(d) sugar substitutes in amounts greater than about 2 mg/ml.

Composition 7, a composition comprising:
(a) water;
(b) acetaminophen in amounts greater than about 50 mg/ml; and
(c) sugar in amounts greater than about 200 mg/ml.

Composition 8, a composition comprising:
(a) water;
(b) acetaminophen in amounts greater than about 50 mg/ml; and
(c) sugar substitutes in amounts greater than about 2 mg/ml.

Composition 9, a composition comprising:
(a) water;
(b) acetaminophen in amounts greater than about 50 mg/ml; and
(c) sugar in amounts greater than about 200 mg/ml; and
(d) sugar substitutes in amounts greater than about 2 mg/ml.

Composition 10, a composition comprising:
(a) water;
(b) bismuth subsalicylate in amounts greater than about 26 mg/ml; and
(c) sugar in amounts greater than about 200 mg/ml.

Composition 11, a composition comprising:
(a) water;
(b) bismuth subsalicylate in amounts greater than about 26 mg/ml; and
(c) sugar substitutes in amounts greater than about 2 mg/ml.

Composition 12, a composition comprising:
(a) water;
(b) bismuth subsalicylate in amounts greater than about 26 mg/ml; and
(c) sugar in amounts greater than about 200 mg/ml; and
(d) sugar substitutes in amounts greater than about 2 mg/ml.

Composition 13, a composition comprising:
(a) water;
(b) caffeine in amounts greater than about 6 mg/ml; and
(c) sugar in amounts greater than about 200 mg/ml.

Composition 14, a composition comprising:
(a) water;
(b) caffeine in amounts greater than about 6 mg/ml; and
(c) sugar substitutes in amounts greater than about 2 mg/ml.

Composition 15, a composition comprising:
(a) water;
(b) caffeine in amounts greater than about 6 mg/ml; and
(c) sugar in amounts greater than about 200 mg/ml; and
(d) sugar substitutes in amounts greater than about 2 mg/ml.

Composition No. 16, any of Compositions 1 to 15 where the composition comprises an encapsulating agent.

Composition No. 17, any of Compositions 1 to 15 where the composition comprises 2 propylene glycol in amounts from about 90 to about 200 mg/ml.

Composition No. 18, any of Compositions 1 to 17 where the composition comprises one or more flavorings.

Composition No. 19, any of Compositions 1 to 17 where the composition has a storage stability of up to 24 months.

Composition No. 20, any of Compositions 1 to 17 where the composition has a storage stability of up to 36 months.

Composition No. 21, any of Compositions 1 to 20 where the composition has a pH of between about 2.0 and about 7.0.

Composition No. 22, any of Compositions 1 to 20 where the composition has a pH greater than about 2.0.

Composition No. 22, any of Compositions 1 to 20 where the composition has a pH less than about 6.0.

While this invention has been disclosed and discussed primarily in terms of specific embodiments thereof, it is not intended to be limited thereto. Other modifications and embodiments will be apparent to the worker in the art.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. A syrup formulation having an over-the-counter active pharmaceutical ingredient, said syrup comprising:
(a) water;
(b) diphenhydramine HCl in amounts greater than about 4 mg/ml; and
(c) sweetener, said sweetener being one or more sweeteners selected from the group consisting of sugars, said sugars being present in amounts greater than about 200 mg/ml, and sugar substitutes, said sugar substitutes being present in amounts greater than about 2 mg/ml.

13. The syrup formulation of claim 12, wherein said diphenhydramine HCl is present in amounts greater than about 10 mg/ml.

14. The syrup formulation of claim 12, wherein said diphenhydramine HCl is present in amounts greater than about 25 mg/ml.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. The syrup formulation of claim 12, wherein said sugars are present in amounts from about 300 to about 700 mg/ml and said sugar substitutes are present in amounts from about 5 to about 20 mg/ml.

69. (canceled)

70. (canceled)

71. (canceled)

72. The syrup formulation of claim 12, wherein said syrup comprises the encapsulating agent propylene glycol in amounts greater than about 90 mg/ml.

73. The syrup formulation of claim 72, wherein said syrup comprises propylene glycol in amounts from about 90 to about 200 mg/ml.

74. (canceled)

75. The syrup formulation of claim 12, wherein said syrup comprises a buffer in amounts sufficient to provide the composition with a pH of between about 2 and about 7.

76. The syrup formulation of claim 12, wherein said syrup comprises a buffer in amounts sufficient to provide the composition with a pH of greater than about 2.

77. The syrup formulation of claim 12, wherein said syrup comprises a buffer in amounts sufficient to provide the composition with a pH of less than about 6.

78. (canceled)

79. (canceled)

80. The syrup formulation of claim 12, wherein said syrup when packaged has a shelf life of at least 12 months.

81. The syrup formulation of claim 12, wherein said syrup when packaged has a shelf life of at least 24 months.

82. The syrup formulation of claim 12, wherein said syrup when packaged has a shelf life of at least 36 months.

83. (canceled)

84. (canceled)

85. A packaged syrup comprising:
(a) a bottle containing about 75 ml or less of the syrup of claim 12;
(b) wherein said syrup provides at least two doses of said diphenhydramine HCl.

86. The packaged syrup of claim 85, wherein said syrup provides at least about 50 said doses.

87. The packaged syrup of claim 83, wherein said syrup provides at least about 100 said doses.

88. A multi-dose packaged syrup, said packaged syrup comprising:
(a) a bottle having a capacity of about 100 ml or less;
(b) at least two doses of the syrup of claim 12 carried within said bottle;
(c) a cap;
(d) a dosage cup releasably carried on said cap; and
(e) a shrink wrap substantially enveloping said bottle and said dosage cup.

89. The packaged syrup of claim 88, wherein said packaged syrup comprises an expanded content label adhered to said shrink wrap.

90. The packaged syrup of claim 88, wherein said expanded content label extends substantially all the way around a vertical wall of said bottle.

91. The packaged syrup of claim 88, wherein said bottle has a capacity of about 75 ml or less.

92. The packaged syrup of claim 88, wherein said bottle has at least about 10 doses of said syrup.
